(19) United States
(12) Patent Application Publication
(10) Pub. No.: US 2026/0007611 A1
(43) Pub. Date: Jan. 8, 2026

(54) ULTRASOUND RESPONSIVE NANOCLUSTER FOR CANCER TREATMENT AND METHOD FOR MANUFACTURING THE SAME

(71) Applicants: INDUSTRY-UNIVERSITY COOPERATION FOUNDATION, YEUNGNAM UNIVERSITY, Gyeongsangbuk-do (KR); INCHEON NATIONAL UNIVERSITY RESEARCH &BUSINESS FOUNDATION, Incheon (KR)

(72) Inventors: Byeong Hee Hwang, Incheon (KR); Jeong Hyun Seo, Gyeongsangbuk-do (KR); Young Hoon Song, Gyeongsangbuk-do (KR); Yeong Chae Ryu, Incheon (KR); Hye Min Cho, Incheon (KR)

(21) Appl. No.: 19/261,281

(22) Filed: Jul. 7, 2025

(30) Foreign Application Priority Data
Jul. 8, 2024 (KR) 10-2024-0089927

(51) Int. Cl. A61K 9/51; A61K 41/00; A61K 45/06; A61P 35/00

(52) U.S. Cl. CPC A61K 9/5115; A61K 9/5161; A61K 41/00; A61K 45/06; A61P 35/00

(57) ABSTRACT

The present invention relates to an ultrasound-responsive nanocluster for cancer treatment and a method for manufacturing the same, the nanocluster according to the present invention has the advantage of efficiently delivering anticancer agents to cancer cells by promoting the release of the drug encapsulated within the nanocluster upon ultrasound treatment.

### CROSS-REFERENCE TO RELATED APPLICATIONS

This application claims the benefit of Korean Patent Application No. KR 10-2024-0089927, filed Jul. 8, 2024, the entire contents of which is incorporated by reference herein.

### TECHNICAL FIELD

The present invention relates to ultrasound responsive nanocluster for cancer treatment and method for manufacturing the same.

### BACKGROUND ART

Breast cancer can be effectively treated when detected at an early stage. However, due to its high recurrence rate and potential to metastasize to other organs such as the liver or lungs, it remains one of the most deadly tumors, accounting for the majority of breast cancer-related deaths. Breast cancer is typically treated with surgical removal of the tumor, followed by systemic or localized chemotherapy to reduce the risk of recurrence. However, clinical application of chemotherapeutic agents is often limited because of their low therapeutic efficacy, non-specific biodistribution, and systemic toxicity.

Doxorubicin (Dox) is an effective drug used to treat various cancers, including breast, ovarian, and lung cancers. Its intrinsic fluorescent properties allow it to be utilized as a versatile tool in research and imaging fields. However, despite its numerous advantages in cancer therapy and diagnostics, it is associated with a significant risk of severe congestive heart failure and cardiomyopathy. To address this issue and minimize the toxicity and drug resistance associated with systemic chemotherapy, various nanoscale drug delivery platforms such as polymeric micelles, polymer-based nanoparticles, and liposomes have been developed to encapsulate doxorubicin. These drug delivery systems contribute to reducing side effects and enhancing drug retention through the enhanced permeability and retention (EPR) effect.

Polymers are useful as carriers in drug delivery systems. Although natural polymers may present challenges in controlling structural and physical properties, they generally exhibit superior biodegradability, biocompatibility, and bioactivity compared to synthetic polymers. In addition, natural polymers with amphiphilic properties have the ability to encapsulate hydrophobic drugs (or hydrophobic nanomaterials) within the core of polymeric nanoparticles or entrap them within a polymer matrix. This ultimately enhances the stability and efficiency of drug delivery. Cancer cells overexpress glucose transporters compared to normal cells, promoting rapid cell proliferation. In particular, breast cancer cells exhibit overexpression of glucose transporter 5 (Glut5). This allows active targeting through polymer structures, especially when interacting with fructose.

The production of nanoparticles using levan is categorized into self-assembly and co-precipitation methods. These methods have been extensively studied for various applications, including macromolecule delivery, anticancer drug delivery, and metal particle coating. However, traditional self-assembly or co-precipitation methods have limitations in forming nanoclusters, which are aggregates of small nanoparticles with uniform size and coating. In addition, for higher therapeutic efficacy, effective drug delivery is required through deeper tissue penetration of the nanocluster and increased release of nanoparticles and drugs from the levan coating. To achieve efficient penetration and release, an external stimulus is needed.

A promising approach involves the non-invasive application of ultrasound (US) to deliver drugs deep into tissues via thermal and physical forces, without the need for surgical intervention. These waves induce localized pressure variations that can generate microbubble cavitation and thermal effects. Cavitation transforms into inertial cavitation, a violent collapse accompanied by shock waves and microstreaming. These localized cavitation effects enhance drug delivery efficiency and therapeutic efficacy by improving nanoparticle scattering, tissue and membrane permeability, and drug retention.

Accordingly, the inventors completed this invention upon confirming that the combination of nanoparticles, chemotherapy, and ultrasound is effective in treating breast cancer.

### RELATED ART DOCUMENTS

### Patent Documents

(Patent Document 001) Korean Patent Application Laid-Open No. 10-2024-0016375 (Feb. 6, 2024)

### DISCLOSURE

### Technical Problem

An object of the present invention is to provide a nanocluster for drug delivery to a cancer cell in a core-shell structure comprising: a core comprising hydrophobic nanoparticles and a hydrophobic drug; and a shell comprising levan.

Another object of the present invention is to provide a method for preventing, ameliorating or treating cancer, comprising administering the nanocluster to a subject in need thereof.

Another object of the present invention is to provide a method for manufacturing a nanocluster, comprising: generating a mixture by combining hydrophobic nanoparticles and a hydrophobic drug; producing nanoclusters by electrospraying the mixture and levan; and delivering the produced nanoclusters to cancer cells by applying ultrasonic treatment.

### Technical Solution

In the present invention, we used an electrospray technique to induce the self-assembly of nanoscale clusters composed of naturally derived levan, hydrophobic silica nanoparticles, and doxorubicin (Dox). Electrospray is effective for the fabrication of nanoparticles and microparticles, as it enables precise size control, uniform dispersion, high encapsulation efficiency, simple processing, and rapid production. Hydrophobic silica nanoparticles can form nanoclusters with hydrophobic doxorubicin through electrospray and be coated with amphiphilic levan. The drug-loaded polymeric nanoclusters were characterized based on their formation of silica cores containing the drug and levan shells, as well as their particle size and stability. Subsequently, we assessed the ultrasound-responsive therapeutic efficacy of the nanoclusters on localized breast tumors and their biocompatibility by injecting them into in vivo mouse xenograft models.

The following description provides a more detailed explanation of the present invention.

The present invention relates to a nanocluster for drug delivery to a cancer cell in a core-shell structure comprising: a core comprising hydrophobic nanoparticles and a hydrophobic drug; and a shell comprising levan.

The nanoclusters of the present invention enable effective drug delivery by enhancing drug stability and preventing burst release, and improve drug delivery efficiency through local accumulation of hydrophobic silica and hydrophobic drugs, ultrasound-triggered release, and enhanced penetration. According to an embodiment of the present invention, the nanocluster was confirmed to be an effective drug carrier capable of delivering a desired amount of drug to the target tissue due to its stability.

In the present invention, the hydrophobic nanoparticles are those coated with fatty acids such as oleic acid and stearic acid or modified with silane, and may comprise one or more selected from a group consisting of SiO₂, Al₂O₃, TiO₂, ZnO, MnO, Mn₂O₃, CuO, FeO, Fe₂O₃, Fe₃O₄, Mn₃O₄, CoO, Co₃O₄, NiO, MgAl₂O₄, CoFe₂O₄, NiFe₂O₄, and CoMn₂O₄.

In the present invention, the hydrophobic drug may be steroids, anti-inflammatory agents or anticancer agents, and may include one or more selected from a group consisting of doxorubicin, sulfasalazine, olsalazine, balsalazide, budesonide, dexamethasone, prednisone, prednisolone, methylprednisolone, hydrocortisone, beclometasone dipropionate, betamethasone, paclitaxel, docetaxel, methotrexate, and indomethacin.

In the present invention, the levan is a naturally occurring fructose polymer, and a biopolymer composed of β-(2→6)-linked fructose units with various degrees of β-(2→1)-linked fructose side chains. The levan exhibits stability under thermal, alkaline, and acidic conditions, and can accumulate at tumor sites through its interaction with glucose transporter 5 (Glut5), a transmembrane protein involved in sugar transport, based on the Warburg effect. Accordingly, the levan was used as a drug delivery carrier for cancer cell treatment in the present invention due to its structure and function.

In the present invention, the core-shell structure may be subjected to ultrasound treatment. The ultrasonic treatment can enhance cellular uptake and drug release of the nanoclusters. In the present invention, the ultrasound treatment may be in a range of 1 to 3 MHz at 0.1 to 2.2 W/cm², preferably in the range of 0.4 to 1.0 W/cm² at 1 MHz, and more preferably in the range of 0.4 to 0.6 W/cm² at 1 MHz.

In the present invention, the core-shell structure may be manufactured by an electrospray method.

In the present invention, the nanocluster may inhibit cell proliferation by enhancing intracellular uptake of the drug via ultrasound stimulation.

In the present invention, the cancer cell may be selected from the group consisting of breast cancer, liver cancer, lung cancer, gastric cancer, rectal cancer, gallbladder cancer, ovarian cancer, bladder cancer, colorectal cancer, lymphoma, brain cancer, uterine cancer, prostate cancer, malignant melanoma, and bile duct cancer.

The cancer cell may include a triple-negative breast cancer cell.

In the present invention, the nanocluster may have an average diameter of 150 to 200 nm. The size of the nanocluster is important to its function, and we confirmed that the size of the nanoclusters is steadily maintained over time by TEM observations. In addition, the electrosprayed L-HSi-Dox nanoclusters according to the present invention exhibited high stability, as they maintained a particle size of less than 300 nm for an extended period in aqueous conditions.

The present invention relates to a pharmaceutical composition for treating cancer, comprising the nanocluster.

In this invention, “treatment” refers to or includes the alleviation, inhibition of progression or prevention of a disease, disorder, pathological condition or one or more symptoms thereof, and “pharmaceutically effective amount” may mean any amount of a composition used in the process of practicing the invention provided herein that is sufficient for the alleviation, inhibition of progression or prevention of a disease, disorder, pathological condition or one or more symptoms thereof.

In the present invention, the method of administering the pharmaceutical composition is not particularly limited and may be administered either orally or non-orally such as via intravenous, subcutaneous, intraperitoneal, inhalation or topical routes, depending on the intended method. The dosage may vary depending on factors such as the patient's body weight, age, sex, health condition, diet, time of administration, route of administration, excretion rate and severity of the disease. The daily dose refers to the amount of the therapeutic substance administered per day that is sufficient to treat the alleviated disease condition in a subject in need of treatment. An effective amount of the therapeutic substance may vary depending on the specific compound, the disease condition and its severity, and the subject in need of treatment, and can be routinely determined by those skilled in the art.

In the present invention, the pharmaceutical composition may include a pharmaceutically acceptable carrier and/or additive. For example, it may include sterile water, physiological saline, conventional buffers (such as phosphate, citrate or other organic acids), stabilizers, salts, antioxidants (such as ascorbic acid), surfactants, suspending agents, isotonic agents or preservatives. For topical administration, the composition may also be combined with organic materials such as biopolymers, inorganic materials such as hydroxyapatite, and specifically, collagen matrices, polylactic acid polymers or copolymers, polyethylene glycol polymers or copolymers, and chemical derivatives thereof. When the pharmaceutical composition according to one embodiment is formulated in an injectable form, the immune cells or substances that enhance their activity may be dissolved in a pharmaceutically acceptable carrier or may be frozen in a solution in which they are dissolved.

In the present invention, the pharmaceutical composition may appropriately include, as needed depending on the route of administration or dosage form, various additives such as suspending agents, solubilizers, stabilizers, isotonic agents, preservatives, anti-adsorption agents, surfactants, diluents, excipients, pH adjusters, analgesics, buffers, reducing agents or antioxidants. Pharmaceutically acceptable carriers and formulations suitable for use in the present invention, including but not limited to those exemplified above, are described in detail in the literature [Remington's Pharmaceutical Sciences, 19th ed., 1995]. The pharmaceutical composition may be formulated into a unit dosage form or filled into a multi-dose container by using pharmaceutically acceptable carriers and/or excipients according to methods that can be readily carried out by those skilled in the art to which the present invention pertains. In this case, the formulation may be in the form of a solution, suspension or emulsion in an oil or aqueous medium or in the form of a powder, granule, tablet or capsule.

In the present invention, the cancer may include breast cancer, liver cancer, lung cancer, gastric cancer, rectal cancer, gallbladder cancer, ovarian cancer, bladder cancer, colorectal cancer, lymphoma, brain cancer, uterine cancer, prostate cancer, malignant melanoma, and bile duct cancer.

The present invention also relates to a method of delivering the nanocluster to cancer cells.

The present invention also relates to a method for preventing, ameliorating or treating cancer, comprising administering the nanocluster to a subject in need thereof.

In the present invention, the cancer may be selected from a group consisting of breast cancer, liver cancer, lung cancer, gastric cancer, rectal cancer, gallbladder cancer, ovarian cancer, bladder cancer, colorectal cancer, lymphoma, brain cancer, uterine cancer, prostate cancer, malignant melanoma, and bile duct cancer.

The cancer may include a triple-negative breast cancer.

In the present invention, the nanocluster may be subjected to ultrasonic treatment.

In the present invention, the ultrasound treatment may be in a range of 1˜3 MHz at 0.1 to 2.2 W/cm², preferably in the range of 0.4˜1.0 W/cm² at 1 MHz, and more preferably in the range of 0.4˜0.6 W/cm² at 1 MHz.

The present invention also relates to a method for manufacturing a nanocluster, comprising: generating a mixture by combining hydrophobic nanoparticles and a hydrophobic drug; producing nanoclusters by electrospraying the mixture and levan; and delivering the produced nanoclusters to cancer cells by applying ultrasonic treatment.

In the present invention, the ultrasound treatment may be in a range of 1˜3 MHz at 0.1 to 2.2 W/cm², preferably in the range of 0.4˜1.0 W/cm² at 1 MHz, and more preferably in the range of 0.4˜0.6 W/cm² at 1 MHz.

In the present invention, the nanocluster may be configured for targeted delivery to a cancer cell selected from the group consisting of breast cancer cell, liver cancer cell, lung cancer cell, gastric cancer cell, rectal cancer cell, gallbladder cancer cell, ovarian cancer cell, bladder cancer cell, colorectal cancer cell, lymphoma, brain cancer cell, uterine cancer cell, prostate cancer cell, malignant melanoma, and bile duct cancer cell.

In the present invention, the nanocluster may be configured for targeted delivery to a triple-negative breast cancer cell.

Meanwhile, the corresponding technical features in the above-described elements may be substituted with those described above, and thus, detailed descriptions thereof are omitted.

### Advantageous Effects

The nanocluster of the present invention enables efficient delivery of anticancer drugs to cancer cells by promoting the release of encapsulated drugs through ultrasound treatment. In addition, the nanocluster of the present invention does not affect the body weight of treated subjects, and thus can be used safely without adverse effects.

### DESCRIPTION OF DRAWINGS

FIG. 1 is a schematic diagram illustrating the composition of the levan nanocluster and its application in a drug delivery system. The levan-shell nanocluster containing doxorubicin was synthesized using an electrospray technique, and the drug was activated by ultrasound for targeted cancer therapy.

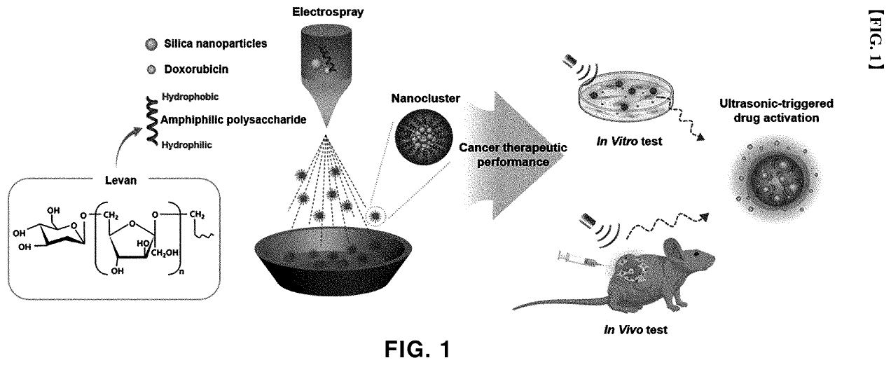

FIG. 2 shows the characterization of the L-HSi-Dox nanocluster manufactured using electrospray. (A) Size distribution determined by dynamic light scattering. (B) Molecular composition analyzed by FTIR spectroscopy. (C) Absorption peak of doxorubicin in the nanocluster measured by UV-vis-NIR spectroscopy. (D) TEM images with various scale bars used to analyze the morphology of the nanocluster. The scale bars are 100 nm for the left and center images, and 20 nm for the right image.

FIG. 3 presents TEM images. (A) hydrophobic silica (HSi), (B) HSi-Dox complex, (C) levan-silica cluster (L-HSi), and (D) doxorubicin-loaded L-HSi cluster (L-HSi-Dox).

FIG. 4 shows high-resolution TEM and EDX line scanning analyses.

FIG. 5 shows the aqueous stability and drug release profile of the L-HSi-Dox nanocluster. (A) Long-term measurement of hydrodynamic size in PBS at 37° C. over 21 days. (B) TEM images of the nanocluster on days 7 and day 21 in PBS at 37° C. (scale bar: 100 nm). (C) Doxorubicin release profiles from the nanocluster in DMSO and water (with and without ultrasound treatment) at 37° C. and 100 rpm for 72 hours. (D) Comparison of doxorubicin release profiles with and without ultrasound treatment in water at 37° C. and 100 rpm for 72 hours. Results are presented as the mean f standard deviation from at least three independent experiments. *p<0.05, ***p<0.005.

FIG. 6 shows the standard curve of free doxorubicin.

FIG. 7 shows the cellular effects of ultrasound-stimulated L-HSi-Dox nanoclusters. (A) Relative cell viability was measured using a CCK assay based on the concentration of L-HSi-Dox nanoclusters. The ultrasound treatment conditions were determined based on (B) ultrasound frequency and intensity, (C) ultrasound intensity at 1 MHz, and (D) timing of ultrasound application. For assessing doxorubicin delivery into cells, fluorescence-based cell counting was performed for (E) untreated cells, (F) cells treated with L-HSi-Dox without ultrasound, and (G) cells treated with L-HSi-Dox with ultrasound. Results are expressed as the mean standard deviation of at least three independent experiments, and each dot represents an independent measurement. Statistical significance is indicated as *, #, **, and *** for p-values of <0.05, 0.05, 0.01, and 0.001, respectively.

FIG. 8 shows cell viability as a function of silica concentration.

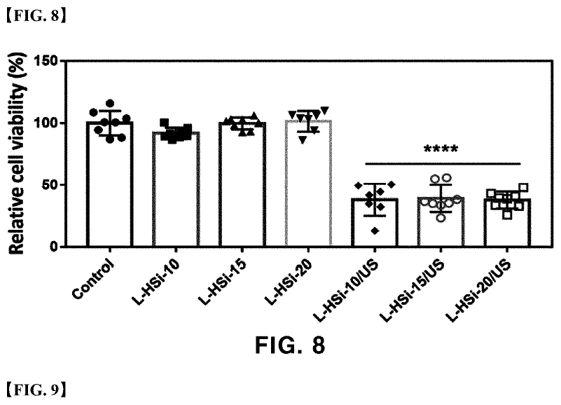

FIG. 9 shows the relative cell viability of various cell lines, including (A) MDA-MB-231 breast cancer cells, (B) HeLa cervical cancer cells, and (C) human neonatal dermal fibroblasts, following treatment with L-HSi and L-HSi-Dox nanoclusters with or without ultrasound (US) stimulation. The nanoclusters were applied under both US-treated and untreated conditions, and cell viability was measured using a CCK assay. The results were normalized to those of the untreated control cells. Data are presented as the mean±standard deviation from at least three independent experiments, with each dot representing an individual result. Statistical significance is indicated as *, **, ***, and **** for p-values of <0.05, 0.01, 0.001, and 0.0001, respectively.

FIG. 10 shows bright-field and fluorescence images of live (green), dead (red), and merged cells treated with L-HSi and L-HSi-Dox nanoclusters and stimulated with ultrasound (A). The number of live cells (B) and the relative cell size (C) under each experimental condition are also presented. Results are shown as the mean±standard deviation from at least three independent experiments, with each dot representing an individual result. Statistical significance is indicated as *, **, ***, and **** for p-values of <0.05, 0.01, 0.001, and 0.0001, respectively.

FIG. 11 shows live cell imaging for quantitative analysis using ImageJ software.

FIG. 12 shows the effect of ultrasound-stimulated L-HSi-Dox nanoclusters in a tumor xenograft mouse model. (A) BALB/c nude mice bearing MDA-MB-231 tumors larger than approximately 30 mm³ received intratumoral injections of L-HSi-Dox every three days, followed by four ultrasound treatments. (B) Images of tumors collected from mice treated with PBS or L-HSi-Dox/US. (C) Relative tumor volumes were measured before each administration. (D) Tumors were harvested on day 18 after treatment, and their weights were measured. (E) Body weight of tumor-bearing mice was monitored throughout the experimental period.

FIG. 13 shows images of mice taken during relative tumor volume measurement.

### MODES OF THE INVENTION

Hereinafter, examples are provided to facilitate understanding of the present invention. However, the following examples are merely illustrative of the present invention and are not intended to limit the scope of the invention. The examples of the present invention are provided to more fully explain the invention to those of ordinary skill in the art.

### 1. Materials and Methods

### 1.1. Materials

Dimethyl sulfoxide (DMSO) and Dox hydrochloride (Dox) were purchased from TCI Chemicals (Tokyo, Japan). N,N-Dimethylformamide (DMF) was purchased from Sigma-Aldrich (St. Louis, MO). Hydrophobic silicon oxide nanoparticles (SiO₂, 5-15 nm) were purchased from SkySpring Nanomaterials Inc. (Houston, TX, USA). The amphiphilic polymer levan was obtained from Real Biotech (Gongju-si, Korea). This study uses levan, a homopolymer of fructose characterized by (2-6) fructofuranosidic linkages, with an average molecular weight of 95 kDa and a degree of polymerization of 587. It is produced commercially through enzymatic and microbial fermentation processes employing Zymomonas mobiliz. All reagents were used as received without further purification. The cell culture medium used was Dulbecco's modified Eagle's medium (DMEM; Corning, New York, MA, USA) supplemented with 10% (v/v) fetal bovine serum (FBS; PAN Biotech, BAV, Germany) and 1% (v/v) penicillin-streptomycin (Life Technologies, Carlsbad, CA, USA). Dulbecco's phosphate-buffered saline (DPBS) and phosphate-buffered saline (PBS) were obtained from GIBCO (Grand Island, NY, USA), while 0.25% trypsin-EDTA (1×) was obtained from Thermo Fisher Scientific (Waltham, MA, USA). The cell culture flasks and plates were obtained from SPL Life Sciences (Gyeonggi-do, South Korea). The D-plus CCK cell viability assay kit from Dongin Biotech (Seoul, South Korea) was used for the WST-1 assay. The LIVE/DEAD Cell Imaging Kit (488/570, cat. R37601) was purchased from Invitrogen (Waltham, MA, USA). Matrigel Matrix for mouse xenograft was procured from Corning (NY, USA).

### 1.2. Fabrication of L-HSi and L-HSi-Dox Nanoclusters

### Preparation of L-HSi Nanoclusters

The L-HSi nanoclusters were prepared by using an electrospray device (eS-robot system, NanoNC, Seoul, Korea). First, 20 mg of hydrophobic silica (HSi) was added to 2 mL of hexane and dispersed by water bath sonication for 20 min. Then, 2 mL of hexane solution with HSi dispersed in 10 mL of levan solution (1.0 wt %) dissolved in DMF. Then, the hydrophobic silica was transferred from the hexane solution in the upper layer to the levan solution in the lower layer by water bath sonication (20 min, 3 times). The mixture dispersed in the DMF phase was recovered and then electrosprayed into an aluminum bath containing water with magnetic stirring. Also, the sprayed nanocluster product was recovered by centrifugation (13,500 rpm, 10 min) and resuspended in water, repeated three times (washing step). Finally, the nanocluster samples dispersed in 20 mL of water were stored in a cold chamber at 4° C. and used within 3 weeks. All procedures were performed at room temperature.

### Preparation of L-HSi-Dox Nanoclusters

The HSi-Dox complex was prepared by adding 2 mg of Dox and 20 mg of hydrophobic silica to 2 mL of hexane and binding Dox to the hydrophobic silica nanoparticles by water bath sonication (20 min, three times). The L-HSi-Dox nanoclusters were prepared in a similar manner except that the HSi-Dox complex was used instead of HSi nanoparticles for drug loading. The encapsulation efficiency (EE %) was calculated according to the following equations.

$$EE ( \% ) = \frac{actual weight of Dox loaded in nanocluster}{initial weight of Dox} \times 100 \% \qquad [ Equation 1 ]$$

### 1.3. Nanocluster Characterization

The morphology of nanoclusters was characterized at magnifications of 10-40 k using a transmission electron microscope (TEM, H-7600, Hitachi, Japan) with a Formvar/carbon 200 mesh copper grid as a holder. 5 μL of nanocluster sample was placed on the TEM grid and dried in a 60° C. oven for 30 min. Elemental analysis of the nanoclusters was performed using a JEM-2200FS TEM (JEOL Co., Japan) with an acceleration voltage of 200 kV. Elemental analysis was performed by energy dispersive X-ray spectroscopy (EDX) line scanning along a linear path passing through the periphery and center of the nanocluster particles. Size distribution and polydispersity index (PDI) of samples transferred to a DTS 1070 capillary cell were determined by dynamic light scattering (DLS) measurement using Zetasizer Nano ZS (Malvern Instruments, USA) equipment. Levan, Dox, HSi-Dox, and L-HSi-Dox nanocluster samples were oven-dried (60° C., overnight) or freeze-dried (FD-1000, Eyela, Japan) and subjected to Fourier transform infrared spectroscopy (FTIR, Spectrum 100 PerkinElmer) measurements in the range of 600 to 4000 cm⁻¹. Also, UV-vis light absorption spectra were measured using an Evolution 201 UV-vis spectrometer (Thermo Scientific). The total amount of nanoclusters was determined by lyophilization, and the Si content was determined by inductively coupled plasma atomic emission spectroscopy (ICP-AES) analysis (Optima 8300, PerkinElmer, USA). The ICP analysis showed that the silica content was 0.367 mg/mL when the total amount of nanoclusters was 1.383 mg/mL.

### 1.4. Dox Release Profile of the Nanocluster

After electrospraying, the nanocluster sample was recovered by centrifugation (13,500 rpm, 10 min), and the process of removing free Dox by resuspension in 50 mL of water was repeated three times (wash step). Finally, nanocluster samples dispersed in 20 mL of water and stored in a 4° C. chamber (to be used within 3 weeks) were tested for Dox release at 37° C. and 100 rpm for a total of 72 h. The L-HSi-Dox clusters were enzymatically degraded using endolevanase from the Megazyme Fructan Assay Kit (40° C., 100 rpm, 3 days) to determine the amount of Dox encapsulated in the nanoclusters. The concentration of Dox in the supernatants was then measured (13,500 rpm, 10 min) using an RF-5301 fluorescence spectrophotometer (Shimadzu, Japan) with excitation and emission wavelengths of 485 and 555 nm, respectively. The concentration of Dox released from the nanoclusters was estimated by comparing the measured fluorescence intensity with the known concentration of free Dox in aqueous solution. The release profile of Dox from the nanoclusters was assessed by measuring the fluorescence of the supernatant separated via centrifugation at 12 h intervals in two liquid mediums (DMSO and water). Next, to determine the Dox release profile of ultrasound-treated nanoclusters, 2 mL samples were placed in a 12-well culture plate and treated at an intensity of 3 MHz and 0.6 W/cm² for 3 min. Ultrasound exposure was applied up to 6 times over 72 h, and the samples collected at 12 h intervals were centrifuged and the fluorescence of the supernatant was measured. Each sample was performed independently. Total incubation was performed at 100 rpm for 72 h at 37° C. The percentage of drug released was calculated using the following equation.

$$drug release ( \% ) = \frac{{Dox}_{f}}{{Dox}_{t}} \times 100 \% \qquad [ Equation 2 ]$$

In Equation 2, Doxₜ represents the total amount of doxorubicin (Dox) measured through enzymatic degradation of the L-HSi-Dox nanocluster, and Dox_{f} represents the amount of Dox released into the medium.

### 1.5. Effective Concentration of L-HSi-Dox Nanoclusters in Cells

An experiment was conducted to determine the effective concentration of L-HSi-Dox using MDA-MB-231 cells. A cell viability assay was performed after an ultrasound applicator (5 cm²) was applied to the center of the four rectangular wells underneath the 96-well plate with the pro-gel. The therapeutic ultrasound machine (Sonicator 740, Mettler Electronics Corp., Anaheim. CA, USA) was set to a frequency of 1 MHz, power density of 0.6 W/cm², pulse width of 50%, and duration of 3 min. The cells were seeded at a density of 1×10⁴ cells/well in a 96-well plate with 100 μL of medium and incubated in a CO₂ incubator for 24 h. After removing 80 μL of the medium, L-HSi-Dox was added to each well at varying Dox levels (69, 138, 277, 553, and 1,106 ng). Fresh medium was added to each well to replenish the total volume of 80 μL. Ultrasound treatment was performed under the same conditions as before (1 MHz, 0.6 W/cm², 3 min), and the plate was incubated in a CO₂ incubator for an additional 2 days. After incubation, 10 μL of a CCK solution from the D-plus CCK cell viability assay kit was added to each well. The absorbance was measured at 450 nm after 2 h using an EPOCH microplate reader.

A cell viability assay was performed to determine the effect of the amount of silica particles in the nanocluster on cell viability. MDA-MB-231 cells were seeded in 100 μL at 1×10⁴ cells per well in a 96-well plate and incubated in a CO₂ incubator for 24 h. The medium in each well was exchanged and L-HSi-Dox (5 μL) with different amounts of silica particles (10, 15, and 20 mg) was added to each well. Ultrasound was then applied under the same conditions as before (1 MHz, 0.6 W/cm², 3 min). The plate was incubated in a CO₂ incubator for another 2 days. Thereafter, 10 μL of CCK solution was added to each well and the absorbance was measured at 450 nm after 2 h using the EPOCH microplate reader.

### 1.6. Optimization of Ultrasonication Conditions and Timing

Two separate CCK assays were performed to optimize ultrasound conditions for minimal cell death and timing. MDA-MB-231 cells were seeded in 100 μL of medium at 1×10⁴ cells per well in a 96-well plate and incubated for 24 h in a CO₂ incubator. The medium was then replaced in each well. To determine the optimal ultrasound condition for minimal cell death, ultrasound was performed for 3 min under six conditions (0.1, 1.1, and 2.2 W/cm² at 1 MHz; and 0.1, 1.1, and 2.2 W/cm² at 3 MHz) for a wide range. Ultrasound was performed for 3 min under four conditions (0.4, 0.6, 0.8, and 1 W/cm² at 1 MHz) for the narrow range. The timing was then tested under three conditions: immediately before treatment with L-HSi-Dox (277 ng of Dox), immediately after treatment, and 4 h after treatment (1 MHz, 0.6 W/cm², 3 min). After sonication, the plate was incubated in a CO₂ incubator for 2 days. For both assays, 10 μL of a CCK solution was added to each well. After 2 h, the absorbance was measured at 450 nm using an EPOCH microplate reader.

1.7. Efficiency of Ultrasound-Assisted Dox Penetration of Nanocluster into Cells

To assess the effectiveness of ultrasound-assisted penetration of Dox-loaded nanoclusters into MDA-MB-231 cells, the cells were seeded in 100 L of culture medium at a density of 1×10⁴ cells per well in a 96-well plate and incubated at 37° C. in a CO₂ incubator for 24 h. L-HSi-Dox was added to each well as previously described, and ultrasound treatment was performed under the same conditions. The L-HSi-Dox with ultrasound was compared to the control condition of L-HSi-Dox without ultrasound. The plate was then incubated in the CO₂ incubator for an additional 2 days. After the medium was removed, 40 μL of trypsin-EDTA was added to each well. The cells were then incubated in a CO₂ incubator for an additional 3 min. Next, the cells were resuspended using a pipet and collected in an E-tube containing 1 mL of DMEM. The collected cells were centrifuged at 300×g for 5 min, and the supernatant was removed. Finally, the cells were resuspended in 50 μL of DPBS and analyzed using a CytoFLEX flow cytometer (Beckman Coulter Korea Ltd., Seoul, South Korea).

### 1.8. Effect of Ultrasound Responsive Nanocluster on Cell Viability

To evaluate the effect of the ultrasound-responsive nanocluster on cell viability, we seeded MDA-MB-231 (human triple-negative breast cancer), HeLa (human cervical cancer), and HDFn (human dermal fibroblast neonatal) cells in 100 μL of DMEM at a density of 1×10⁴ cells per well in a 96-well plate. In the cell-only condition, 80 L of medium was replaced with 80 μL of fresh DMEM the next day. For cells receiving L-HSi-Dox and L-HSi-Dox, each nanocluster solution containing 277 ng Dox was added to 75 μL of fresh DMEM. Ultrasonic treatment was performed under the optimal conditions of 1 MHz and 0.6 W/cm² for 3 min immediately after the addition of nanoclusters with 3 min incubation intervals between treatments. After ultrasound treatment, the cells were incubated for 2 days in a CO₂ incubator. The L-HSi-Dox with ultrasound was compared to the control condition of L-HSi-Dox without ultrasound. The CCK solution (10 μL) was added to each well of the plate, which was then covered with aluminum foil (Daihan, Cheonan, South Korea) and incubated for 2 h. The absorbance was measured at 450 nm by using an EPOCH microplate reader (BioTek Instruments, Inc. Winooski, VT, USA).

1.9. Live and Dead Cell Assay after L-HSi-Dox and Ultrasound Treatment

To evaluate the cytotoxicity of L-HSi-Dox and ultrasound treatments, MDA-MB-231 cells were seeded at a density of 1×10⁴ cells/well in 96-well plates and incubated in a CO₂ incubator for 24 h. L-HSi-Dox (277 ng of Dox) was added to each well as previously described, and ultrasound treatment was performed under the same conditions. The plate was incubated for 2 days in a CO₂ incubator. After medium removal, 25 μL of Opti-MEM was added to each well, followed by 25 μL of a mixture of component A and component B (LIVE/DEAD solution). The plate was wrapped in aluminum foil and incubated for 20 min in a CO₂ incubator. Fluorescence images were captured by using a fluorescence microscope (Nikon ECLIPSE Ti2; Nikon, Tokyo, Japan) to assess cell viability.

### 1.10. In Vivo Xenografted Mouse Experiments

All procedures involving animals were conducted in strict adherence to EU Directive 2010/63/EU on the protection of animals used for scientific purposes and were approved by the Institutional Animal Care and Use Committee (IACUC) of Incheon National University under the approval number INU-ANIM-2022-04. To prepare the tumor xenograft mice, 6-week-old female BALB/c nude mice were anesthetized with 2% isoflurane and 99.99% oxygen gas using a VetFlo traditional anesthesia system (Kent Scientific Corp., Torrington, CT, USA). MDA-MB-231 cells originating from human female breast cancer, are typically xenografted into female mice due to their relevance to the female hormonal and biological environment. To initiate the xenograft, we gently mixed 10⁷ MDA-MB-231 cells with 50 μL of PBS and an equal volume of Matrigel.

The resulting cell mixture was subcutaneously injected on both sides of the back using a 1 mL insulin syringe (Becton, Dickinson, and Company, Franklin Lakes, NJ, USA). When the tumor volume reached at least 30 mm³, we administered 27,650 ng of Dox (10 μL) within the nanoclusters intratumorally to the mice using a 0.3 mL insulin syringe. The L-HSi-Dox-injected areas were then exposed to an applicator loaded with ultrasound transmission gel. Ultrasonication was performed for 7 min at 1 MHz, 1.6 W/cm², and 50% duty cycle (1 min on/30 s off) using a Sonicator 740. L-HSi-Dox injection and ultrasonication were repeated every 3 days. For a total of 4 cycles, the tumor volumes were measured using calipers every 3 days and calculated using the formula: 0.52×length (mm)×width (mm)×height (mm). After sacrificing the mice, their tumors were harvested and weighed using a microbalance (OHAUS, NJ, USA).

### 1.11. Statistical Analysis

Quantitative data were obtained from at least three independent experiments and presented as mean±standard deviation. A statistical analysis was performed using Excel's statistical functions. The first step involved conducting an F-test to evaluate the equality of variances between the two groups. A p-value greater than 0.05 indicated homogeneity of variances, while a value below 0.05 indicated heterogeneity. Therefore, a t test was performed based on the F-test results. The graphical representation denotes the significance levels from the t test as follows: *p<0.05, **p<0.01, and ***p<0.001.

### 2. Results

### 2.1. Nanocluster Construction and Characterization

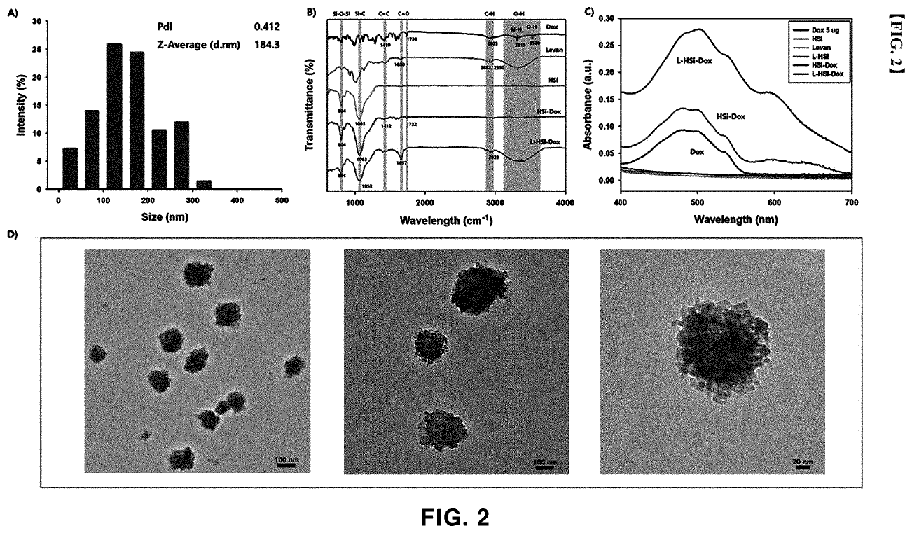

Levan-based nanoclusters were created using a method similar to that used in a previous study (Park, J. C.; Kim, D. H.; Song, Y. H.; Cha, H. J.; Seo, J. H. ACS Appl. Mater. Interfaces 2020). Briefly, HSi (2 mg/mL) and Dox (200 μg/mL) were prepared as HSi-Dox complexes in hexane by using ultrasonication. The HSi-Dox complex was then added to a 1 wt % levan solution dissolved in DMF and sonicated for approximately 1 h. A mixture of levano and hydrophobic HSi-Dox complexes in DMF was electrosprayed into a water bath. The L-HSi-Dox nanoclusters were fabricated successfully by using electrospraying. The Z-average diameter was approximately 184 nm (PDI:0.412) (FIG. 2A).

The FT-IR spectra (FIG. 2B) of silica, levan, and L-HSi-Dox nanoclusters show prominent peaks, indicating successful integration of each material into the nanoclusters. The peaks at 804 and 1063 cm⁻¹ in the spectra of the silica nanoparticles correspond to the Si—O—Si stretching and siloxane vibrations of the (SiO)n group, respectively. The broad stretching vibrations at ˜3,300 cm⁻¹ are characteristic of the hydroxyl (—OH) groups of levan or adsorbed water molecules. The IR spectrum of Dox alone shows characteristic absorption peaks that correspond to the vibrations of the different functional groups. These include N—H stretch (3,310 cm⁻¹), C═O stretch (1,730 cm⁻¹), and C═C ring stretch (1410 cm⁻¹), which were clearly observed.

UV-vis spectroscopy confirmed the presence of Dox in the nanoclusters. FIG. 2C shows the absorbance peaks in the presence and absence of Dox. The absorbance of free Dox exhibited a broad peak at 410-570 nm. Similarly, only the HSi-Dox complexes and L-HSi-Dox nanoclusters containing Dox had broad peaks in the same range. Therefore, the successful synthesis of L-HSi-Dox nanoclusters was confirmed through the integration of the levan biopolymer and HSi-Dox complex.

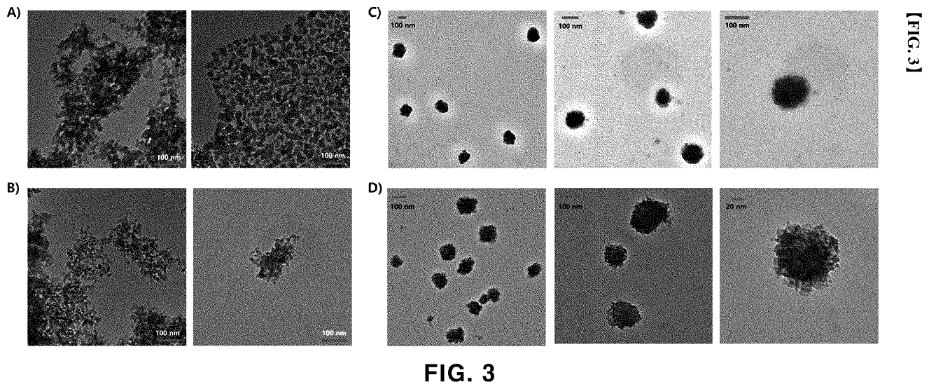

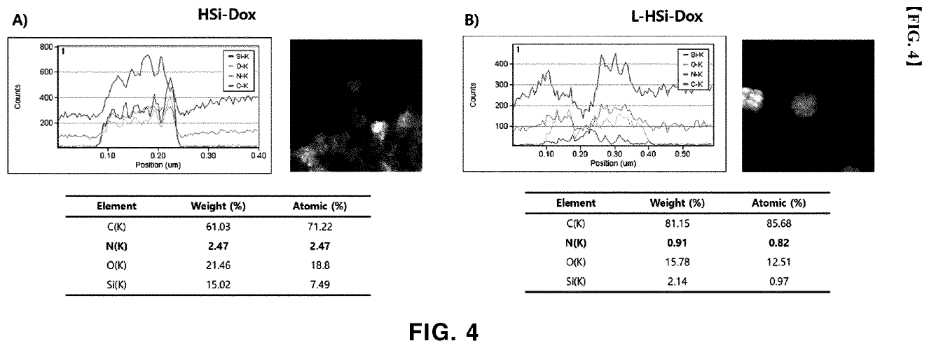

Furthermore, the TEM images revealed the morphology of the nanocluster (FIG. 2D, FIG. 3). The transmission electron microscopy (TEM) images revealed that the nanocluster maintained a spherical morphology and particle size range, consistent with the dynamic light scattering (DLS) data. The energy-dispersive X-ray (EDX) line scanning spectra displayed peaks for silicon (Si), carbon (C), oxygen (O), and nitrogen (N), which may have originated from silica, levan, and Dox (FIG. 4). In the presence of the levan biopolymer, the C content of the nanocluster increased compared with that of the HSi-Dox complex. Additionally, the N, O, and Si contents decreased, indicating the formation of the L-HSi-Dox nanoclusters.

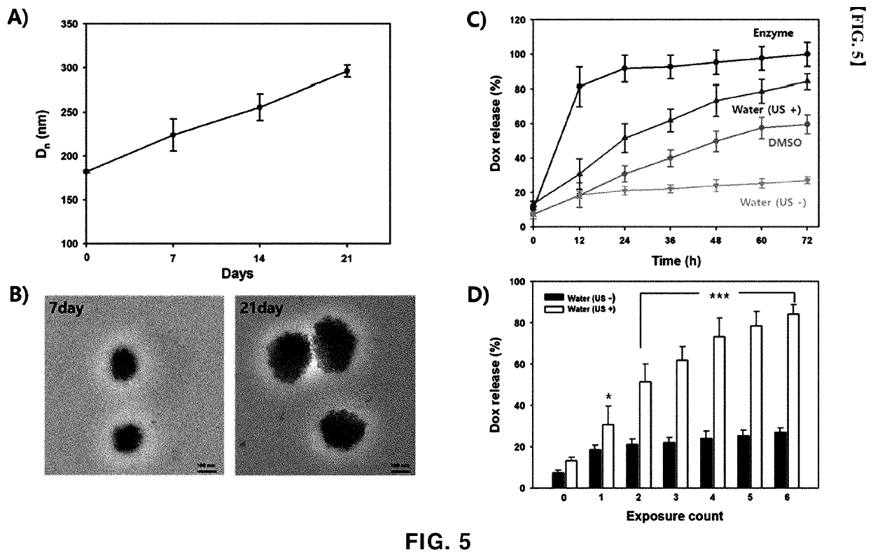

FIG. 5A, FIG. 5B shows the results of the evaluation of nanocluster aqueous stability using size distribution analysis and TEM analysis. The evaluation was performed in PBS solution at 37° C. for 3 weeks. The hydrodynamic size of the nanoclusters in aqueous solution appears to have increased slightly from 181 to 291 nm over 3 weeks due to swelling. Additionally, the polydispersity index (PDI) increased from 0.48 to 0.64 after 3 weeks in water. The PDI value increase suggests that the sample size distribution increased or agglomerated over time compared to its initial state.

### 2.2. Dox Release Profile and Encapsulation Efficiency of Nanocluster

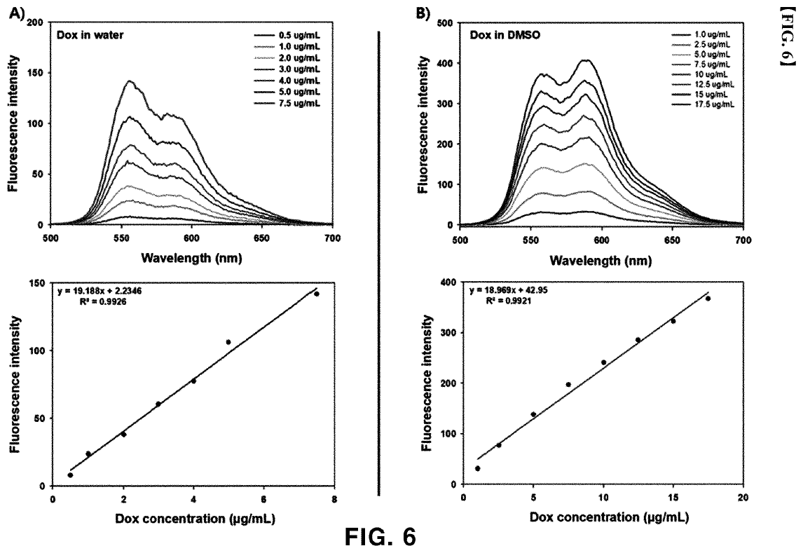

FIG. 5C and FIG. 6 display the release profiles of Dox in water and DMSO (Table 1). The profile was determined based on the total amount of Dox (55.3 μg/mL, line 1) obtained through enzymatic degradation of the nanoclusters. The initial release values of Dox from the L-HSi-Dox nanoclusters were 30.86% (line 3) and 21.23% (line 4) within 24 h in DMSO and water, respectively. It is worth noting that during the 72 h period, only 27.16% of Dox was released in the aqueous medium. Excluding the initial release in the first 24 h, only 5.93% of Dox was released. In contrast, the release of 59.68% of Dox in the DMSO medium may have resulted in a relatively greater Dox release than that achieved in the water condition.

TABLE 1

| | Dox release | | | |
|---|---|---|---|---|
| | DMSO | | Water | |
| Time | (μg/mL) | (%)a | (μg/mL) | (%)a |
| 0 h | 4.10 ± 1.39 | 7.42 ± 2.52 | 4.19 ± 0.72 | 7.59 ± 1.31 |
| 24 h | 17.05 ± 2.62 | 30.86 ± 4.74 | 11.73 ± 1.37 | 21.23 ± 2.47 |
| 48 h | 27.56 ± 3.36 | 49.88 ± 6.09 | 13.33 ± 1.94 | 24.13 ± 3.52 |
| 72 h | 32.98 ± 3.02 | 59.68 ± 5.47 | 15.01 ± 1.12 | 27.16 ± 2.03 |

Table 1. The amount of doxorubicin (Dox) released from the L-HSi-Dox nanocluster. The release percentage (%) of Dox was determined based on the total amount of Dox (55.3 μg/mL).

FIG. 5C and FIG. 5D shows the release of ultrasound-treated nanoclusters. As shown, ultrasound-exposed nanoclusters showed a significant increase in Dox release compared to untreated samples (FIG. 5C, lines 2 and 4). With an increasing number of ultrasound exposures and over time, samples achieved triggered drug release ranging from 30.83% at 12 h (1 exposure) to 84.35% at 72 h (6 exposures) samples (FIG. 5D). This represents a superior release effect compared to the release of ultrasound-untreated nanoclusters (27.16%) during 72 h.

The release profile was used to evaluate the encapsulation efficiency (EE %) of 1-HSi-Dox nanoclusters prepared by electrospray. Out of a total of 200 μg of Dox, 110.6 μg were loaded into the nanocluster, resulting in an encapsulation efficiency (EE %) of 55.3%.

### 2.3. Impact of Sonication on Dox Delivery Efficiency

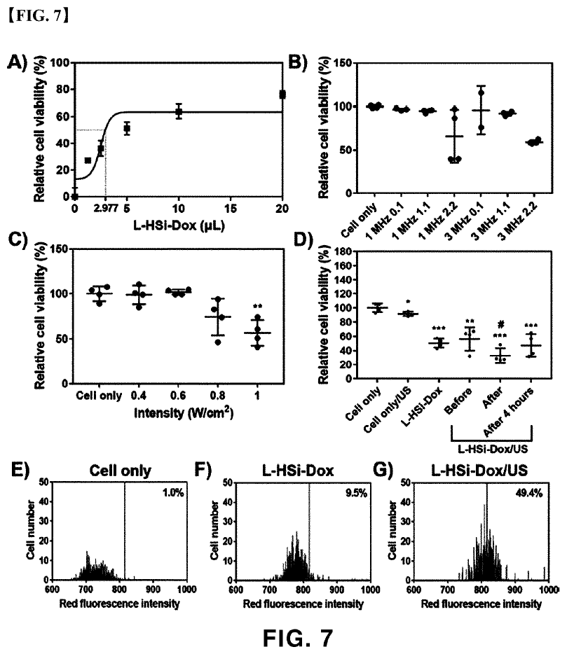

Measuring the Dox content within the L-HSi-Dox nanoclusters accurately was difficult due to quenching, as shown in FIG. 5. To determine the optimal quantity of nanoclusters for cell experiments, we examined the effect of the quantity of L-HSi-Dox nanoclusters on cell viability (FIG. 7A). Our findings revealed a consistent trend of decreasing cell viability as the number of nanoclusters increased. A 50% decrease in cell viability was observed with a quantity of 164.6 ng. Therefore, the L-HSi-Dox nanocluster was used at the scale of several hundred Dox for subsequent experiments.

To determine the optimal ultrasound conditions for the experiment, we explored variations in the frequency and intensity (refer to FIG. 7B). Our findings indicate that frequencies of 1 and 3 MHz did not result in a significant decrease in cell viability up to 1.1 W/cm². However, a statistically significant reduction of 59.2% was observed at 2.2 W/cm² (p-value=1.1×10⁻⁷). Additionally, a slightly smaller decrease in cell viability was observed at 1 MHz compared to that at 3 MHz. This prompted further experiments to establish more precise ultrasound conditions within the range of 0.4 to 1.0 W/cm² at 1 MHz (FIG. 7C). Based on these experiments, the cell viability remained stable up to 0.6 W/cm² but significantly decreased to 74.2 and 57.0% at 0.8 and 1.0 W/cm², respectively (p-values=0.056, 0.0018). Subsequent experiments were conducted under ultrasonic conditions of 1 MHz and 0.6 W/cm².

To determine the optimal timing of ultrasound application, cell viability was measured immediately before treatment with L-HSi-Dox nanoclusters, immediately after treatment, and 4 h after treatment, resulting in cell viabilities of 56.4, 32.8, and 47.3%, respectively (FIG. 7D, p=0.0024, 3.2×10⁻⁵, 0.00081). Application of ultrasound immediately after nanocluster treatment was found to be the most effective approach to reduce cell viability. As a result, this timing was used in subsequent experiments. Through these experiments, conditions were established to minimize the impact of ultrasound on the cells while maximizing the synergistic effect between the L-HSi-Dox nanoclusters and ultrasound.

Flow cytometry was used to evaluate the efficacy of L-HSi-Dox nanoclusters in delivering Dox to cells following sonication. Using the top 1% of the fluorescence intensity in untreated cells as a reference baseline (FIG. 7E), we observed a 9.5% increase in the Dox-positive cell population in the absence of sonication, whereas sonication resulted in a significant 49.4% increase (FIG. 7F, FIG. 7G). The mean fluorescence intensities of the cell-only, L-HSi-Dox, and L-HSi-Dox/US groups were 728.1, 793.9, and 821.5, respectively.

2.4. Cell-Line-Dependent Viability Affected by L-HSi-Dox Nanocluster with Sonication

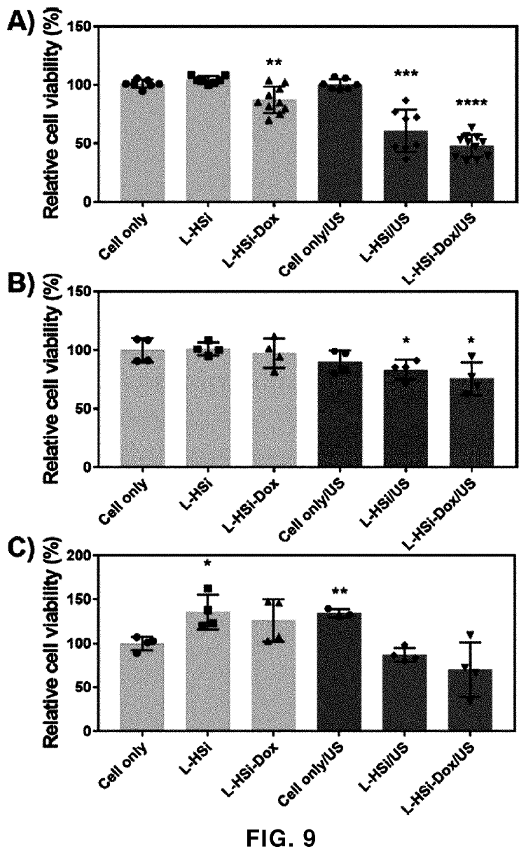

A CCK assay was performed to evaluate the effects of L-HSi-Dox and ultrasound treatment on different cell lines (FIG. 9). Relative cell viabilities were measured under different conditions, including cell-only, L-HSi, L-HSi-Dox, cell-only/US, L-HSi/US, and L-HSi-Dox/US. In the MDA-MB-231 cell line (FIG. 9A), relative cell viability did not differ significantly between cells without and with ultrasound under the cell-only condition. However, the L-HSi and L-HSi-Dox nanoclusters caused a significant decrease in cell viability (60.3% and 47.5%, respectively) after sonication (p=0.0003 and 1.67×10⁻¹⁰, respectively). Notably, L-HSi-Dox decreased cell viability (86.5%) even without sonication (p=0.0041).

For the human cervical cancer cell line HeLa (FIG. 9B), the relative cell viabilities (83.3 and 75.7%, respectively) were reduced for cells treated with the L-HSi and L-HSi-Dox nanoclusters after sonication (p=0.0461 and 0.0307, respectively); however, the degree of reduction was less than that observed for the MDA-MB-231 cell line. For the neonatal normal human dermal fibroblast cell line HDFn (FIG. 9C), the relative cell viabilities were higher than those of the cancer cell lines under all conditions, and the degree of decrease in relative cell viabilities (87.2 and 70.3%, respectively) after ultrasonication with L-HSi and L-HSi-Dox was less than that observed for the cancer cell lines. These results suggest that the effect of ultrasonicated L-HSi-Dox nanoclusters on cell viability was dependent on cell type, with cancer cells being more sensitive to treatment than normal cells.

### 2.5. Ultrasound-Stimulated Nanoclusters and Dox Delivery-Induced Cancer Cell Death

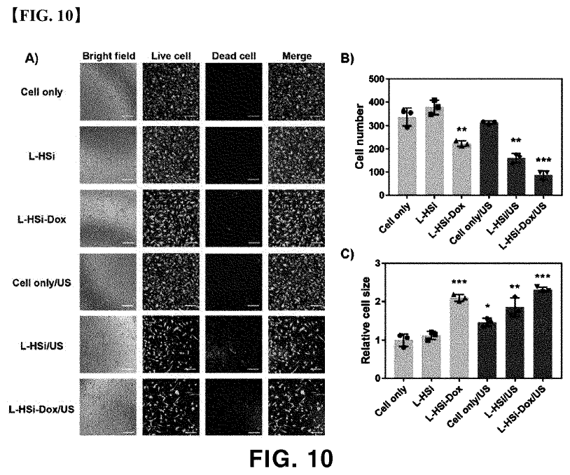

To evaluate the effect of ultrasound treatment on the anticancer effects of L-HSi and L-HSi-Dox on MDA-MB-231 cells, live and dead cell assays were performed (FIG. 10A). The staining protocol included the use of calcein-AM, which fluoresces green upon enzymatic conversion in live cells, and iodide BOBO-3, which stains DNA outside the cell membranes of dead cells. Of note, images of the cell-only and cell/ultrasound conditions showed minimal or no DNA staining. However, some dead cells were observed in the images of cells treated with L-HSi and L-HSi-Dox even without ultrasound, while with ultrasound, the nanoclusters showed a significant decrease in green fluorescence and a significant increase in red fluorescence. The larger size and reduced number of live cells in the L-HSi-Dox group compared with the L-Hsi group suggest that Dox may have inhibited cell division. Taken together, these results indicate that ultrasound treatment enhances the effect of nanoclusters on cells, resulting in increased cell death, and induces an even more pronounced effect when combined with Dox.

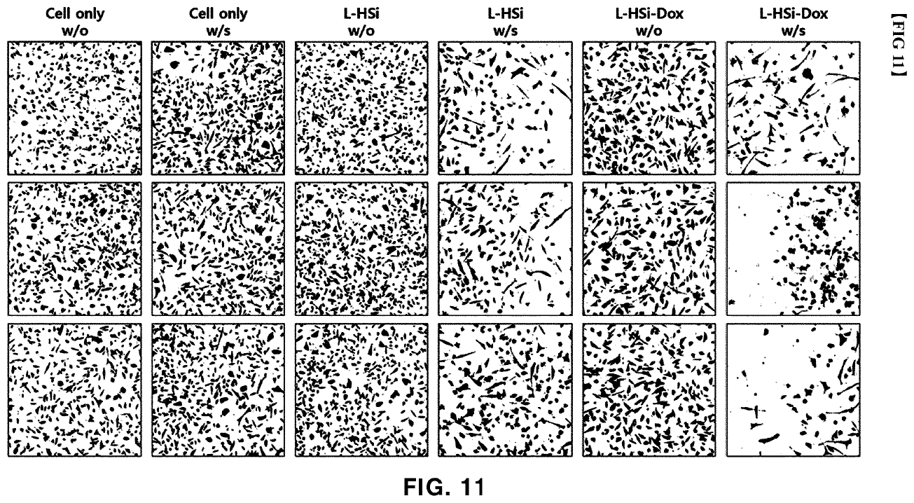

Quantitative analysis of the images revealed a decrease in the number of MDA-MB-231 cells after exposure to ultrasound or after the addition of Dox to the nanoclusters (FIG. 10B, FIG. 11 and Table 2). The application of ultrasound to L-HSi-Dox resulted in a statistically significant and largest reduction in cell number (74.4%, p=0.0005). The addition of L-HSi-Dox resulted in a 34% reduction in cell number (p=0.007). While the combination of L-HSi with ultrasound resulted in a 52.4% reduction (p=0.002). Exposure to ultrasound or the addition of Dox to the nanoclusters resulted in an increase in cell size (FIG. 10C). In particular, the application of ultrasound to L-HSi-Dox resulted in a statistically significant increase in cell size (131%, p-value=0.00021). The addition of L-HSi-Dox resulted in a 109% increase in cell size (p-value=0.0005), while the combination of L-HSi with ultrasound resulted in an 85% increase (p-value=0.007). Although no significant decrease in the cell number was observed with ultrasound application, a significant increase in cell size of 46% (p-value=0.014) was observed. In conclusion, combined treatment with the nanocluster and ultrasound decreased the cell number and increased cell size by inhibiting cell division. Furthermore, the addition of Dox to this combination enhanced these effects.

TABLE 2

| | Count | Total Area | Average Size | % Area |
|---|---|---|---|---|
| Cell only-w/o | 361 | 593747 | 1.00 | 14.156 |
| Cell only-w/s | 305 | 702667 | 1.40 | 16.753 |
| L-HSi-w/o | 349 | 779290 | 1.36 | 18.58 |
| L-HSi-w/s | 138 | 446786 | 1.97 | 10.652 |
| L-HSi-Dox-w/o | 209 | 766917 | 2.23 | 18.285 |
| L-HSi-Dox-w/s | 97 | 405002 | 2.54 | 9.656 |

Table 2. Quantitative analysis of live cell images using ImageJ software.

### 2.6. Antitumor Effects of Nanoclusters and Ultrasound Treatment

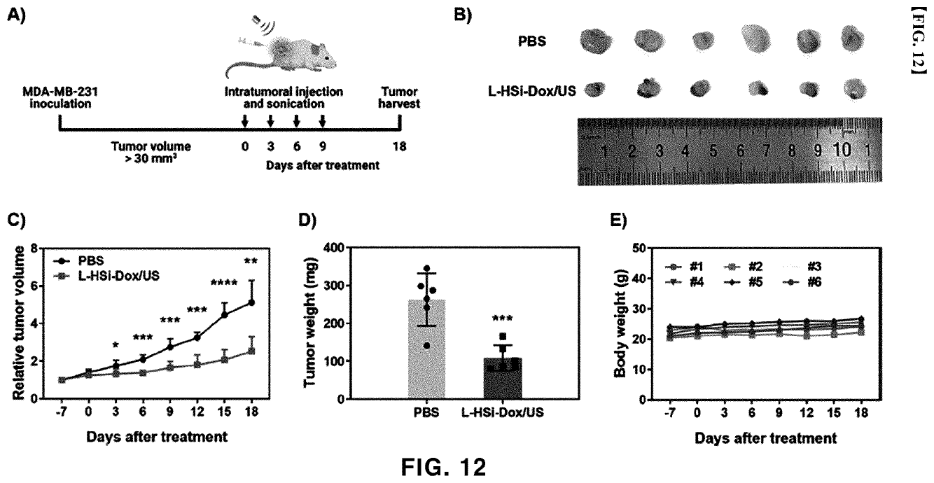

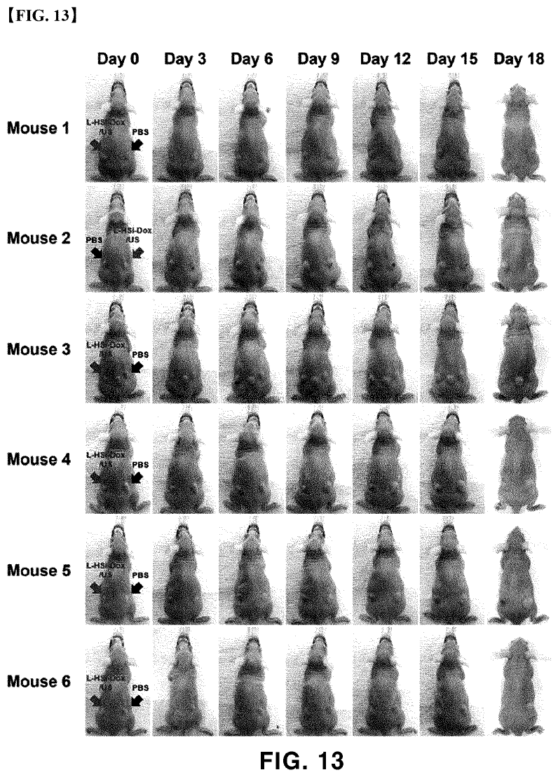

A human breast tumor xenograft mouse model was used to evaluate the anticancer effects of our L-HSi-Dox nanoclusters and ultrasound (FIG. 12 and FIG. 13). In the tumor-xenografted mice, nanocluster and ultrasound treatment significantly reduced the relative tumor volumes compared to PBS treatment and reduced the average tumor weight by 2.4-fold while maintaining constant body weight. The experimental animal model is shown in FIG. 12A. Briefly, L-HSi-Dox and PBS were administered intratumorally to left and right MDA-MB-231 tumors of approximately 30 mm³ or larger in BALB/c nude mice with ultrasound at 3 day intervals for a total of 4 treatments. The ultrasound conditions used in the animal studies were safe, and no differences in appearance between normal and treated tissues were observed. Conditions were chosen to produce a moderate increase in the temperature after treatment.

Tumor size was measured before each treatment, and relative tumor volume was calculated based on the measurements taken before the first treatment (FIG. 12B and FIG. 12C). The relative tumor volumes treated with L-HSi-Dox/US increased very slowly, up to approximately 2-fold relative to the initial volume, whereas the tumor volumes treated with PBS increased very rapidly over time, up to approximately 5-fold. After the first treatment, a statistically significant difference was found between the PBS and L-HSi-Dox/US groups at each time point (p-values of 0.023, 0.00012, 0.00069, 0.00013, 0.00004, and 0.0010). The tumor weight on day 18 after initial treatment with L-HSi-Dox/US was significantly reduced by 2.4-fold compared to PBS treatment (FIG. 12D, p=0.00062). Throughout the treatment period, tumor-xenografted mice maintained a constant body weight (FIG. 12E).

While the specific aspects of the present invention have been described in detail above, it will be apparent to those skilled in the art that such detailed descriptions are merely preferred embodiments, and the scope of the present invention is not limited thereto. Accordingly, the true scope of the present invention shall be defined by the appended claims and their equivalents.

What is claimed is:

1. A nanocluster for drug delivery to a cancer cell in a core-shell structure comprising:
a core comprising hydrophobic nanoparticles and a hydrophobic drug; and a shell comprising levan.

2. The nanocluster of claim 1, wherein the hydrophobic nanoparticles are one or more selected from a group consisting of SiO₂, Al₂O₃, TiO₂, ZnO, MnO, Mn₂O₃, CuO, FeO, Fe₂O₃, Fe₃O₄, Mn₃O₄, CoO, Co₃O₄, NiO, MgAl₂O₄, CoFe₂O₄, NiFe₂O₄, and CoMn₂O₄.

3. The nanocluster of claim 1, wherein the hydrophobic drug is one or more steroids, anti-inflammatory agents or anticancer agents selected from a group consisting of doxorubicin, sulfasalazine, olsalazine, balsalazide, budesonide, dexamethasone, prednisone, prednisolone, methylprednisolone, hydrocortisone, beclometasone dipropionate, betamethasone, paclitaxel, docetaxel, methotrexate, and indomethacin.

4. The nanocluster of claim 1, wherein the nanocluster is subjected to ultrasonic treatment.

5. The nanocluster of claim 4, wherein the ultrasonic treatment is in a range of 0.1 to 2.2 W/cm² at 1 to 3 MHz.

6. The nanocluster of claim 1, wherein the core-shell structure is manufactured by an electrospray method.

7. The nanocluster of claim 1, wherein the nanocluster inhibits cell proliferation by enhancing intracellular uptake of the drug via ultrasound stimulation.

8. The nanocluster of claim 1, wherein the cancer cell is selected from the group consisting of breast cancer cell, liver cancer cell, lung cancer cell, gastric cancer cell, rectal cancer cell, gallbladder cancer cell, ovarian cancer cell, bladder cancer cell, colorectal cancer cell, lymphoma, brain cancer cell, uterine cancer cell, prostate cancer cell, malignant melanoma, and bile duct cancer cell.

9. The nanocluster of claim 1, wherein the cancer cell includes a triple-negative breast cancer cell.

10. The nanocluster of claim 1, wherein the nanocluster has an average diameter of 150 to 200 nm.

11. A method for preventing, ameliorating or treating cancer, comprising administering the nanocluster of claim 1 to a subject in need thereof.

12. The method of claim 11,
wherein the cancer is selected from a group consisting of breast cancer, liver cancer, lung cancer, gastric cancer, rectal cancer, gallbladder cancer, ovarian cancer, bladder cancer, colorectal cancer, lymphoma, brain cancer, uterine cancer, prostate cancer, malignant melanoma, and bile duct cancer.

13. The method of claim 11,
wherein the cancer includes a triple-negative breast cancer.

14. The method of claim 11,
wherein the nanocluster is subjected to ultrasonic treatment.

15. The method of claim 14,
wherein the ultrasonic treatment is in a range of 0.1 to 2.2 W/cm² at 1 to 3 MHz.

16. A method for manufacturing a nanocluster, comprising:
generating a mixture by combining hydrophobic nanoparticles and a hydrophobic drug;
producing nanoclusters by electrospraying the mixture and levan; and
delivering the produced nanoclusters to cancer cells by applying ultrasonic treatment.

17. The method of claim 16, wherein the ultrasonic treatment is in a range of 0.1 to 2.2 W/cm² at 1 to 3 MHz.

18. The method of claim 16, wherein the nanocluster is configured for targeted delivery to a cancer cell selected from the group consisting of breast cancer cell, liver cancer cell, lung cancer cell, gastric cancer cell, rectal cancer cell, gallbladder cancer cell, ovarian cancer cell, bladder cancer cell, colorectal cancer cell, lymphoma, brain cancer cell, uterine cancer cell, prostate cancer cell, malignant melanoma, and bile duct cancer cell.

19. The method of claim 16, wherein the nanocluster is configured for targeted delivery to a triple-negative breast cancer cell.
